(19) United States
(12) Patent Application Publication
(10) Pub. No.: US 2023/0324357 A1
(43) Pub. Date: Oct. 12, 2023

(54) System And A Method For Determining Characteristics Of Food

(71) Applicant: Isofoodtrace Limited, Hong Kong (HK)

(72) Inventors: Chung-Lim LUK, Hong Kong (HK); Inga E. CONTI-JERPE, San Francisco, CA (US); Wendy F. C. MCLEOD, Hong Kong (HK)

(21) Appl. No.: 18/131,461

(22) Filed: Apr. 6, 2023

Related U.S. Application Data
(60) Provisional application No. 63/328,375, filed on Apr. 7, 2022.

(51) Int. Cl. G01N 33/02; G01N 1/44

(52) U.S. Cl. CPC G01N 33/02; H01J 49/26; G01N 1/44

(57) ABSTRACT

The invention provides a method of determining a characteristic of a food substance. The method comprises detecting at least one stable isotope of one or more elements of a food substance with unknown characteristic; processing signal received from detecting the at least one stable isotope of the one or more elements; analysing the signal processed with respect to a standard isotopic profile of the food substance with known characteristic thereby deducing a characteristic of the food substance being tested. The invention further provides a system implementing the method above-described.

### CROSS-REFERENCE TO RELATE APPLICATIONS

This application claims the benefit of U.S. Application 63/328,375, filed Apr. 7, 2022, the contents of which are incorporated herein by reference.

### FIELD OF THE INVENTION

The invention relates to the field of food verification and authentication. Particularly but not exclusively, the invention relates to the verification and authentication on characteristics of food based on isotope analysis.

### BACKGROUND OF THE INVENTION

There have been growing interests from the food industry, the commercial and the general public on the authentication of food, and particularly, verification on characteristics or claims of food products such as, but are not limited to, ingredients, components, properties, natures, geographic origin and other qualities of the substance comprising the food products. In general, information of food product can be identifiable from the labeling and/or packaging of the product, especially for premium high-quality food products such as beef, wine, fruits and honey, etc. Depending on the product type, information such as geographic origin, i.e., where the produce was grown or the livestock was born; and production or farming practice such as whether the livestock was grain-fed or grass-fed, whether the poultry was caged or free-ranged, or whether the seafood is farmed or wild caught, etc. could be made available to the consumer by the food manufacturers. Nevertheless, in many countries the disclosure of food product information is still voluntary, and even if the information is available to the public, the product claims may not be regulated and therefore, the information receivable by the consumers could be incomplete, misleading or mistaken.

It is therefore beneficial for the food information to be more accessible to the general consumers with accuracy. However, traditional food verification methods are rather limited, and in most situations, geographic details of a food product can only be traced through the supply chain. Whether the claimed qualities are genuine may only be verified by analytical techniques which are known to the complicated, expensive and generally lacking of specificity.

### OBJECTS OF THE INVENTION

An object of the present invention is to provide a system and a method for food verification based on isotope analytic.

Another object of the present invention is to mitigate or obviate to some degree one or more problems associated with known food verification or authentication technique, or at least to provide a useful alternative.

The above objects are met by the combination of features of the main claims; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

In a first main aspect, the invention provides a method of determining a characteristic of a food substance. The method comprises detecting at least one stable isotope of one or more elements of a food substance with unknown characteristic; processing signal received from detecting the at least one stable isotope of the one or more elements; analysing the signal processed with respect to a standard isotopic profile of the food substance with known characteristic thereby deducing a characteristic of the food substance being tested.

In a second main aspect, the invention provides a system implementing steps of the first main aspect for determining a characteristic of a food substance. The system comprises an apparatus for detecting signal of at least one stable isotope of one or more elements of the food substance with unknown characteristic; and a computer device adapted to receive signal of at least one stable isotope of one or more elements of the food substance detected from the apparatus, the computer device comprising a memory for storing data and a processor for executing computer readable instructions; wherein the processor is configured by the computer readable instructions when being executed thereby implement steps of processing the signal detected, and analysing the processed signal with respect to a standard isotopic profile of the food substance with known characteristic thereby deducing a characteristic of the food substance being tested.

The summary of the invention does not necessarily disclose all the features essential for defining the invention; the invention may reside in a sub-combination of the disclosed features.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 is a schematic diagram showing a method for determining a characteristic of a food substance according to an embodiment of the present invention;

FIG. 2 is a schematic diagram showing the detection of stable isotopes of elements comprising the food substance by a mass spectrometer according to the method of FIG. 1;

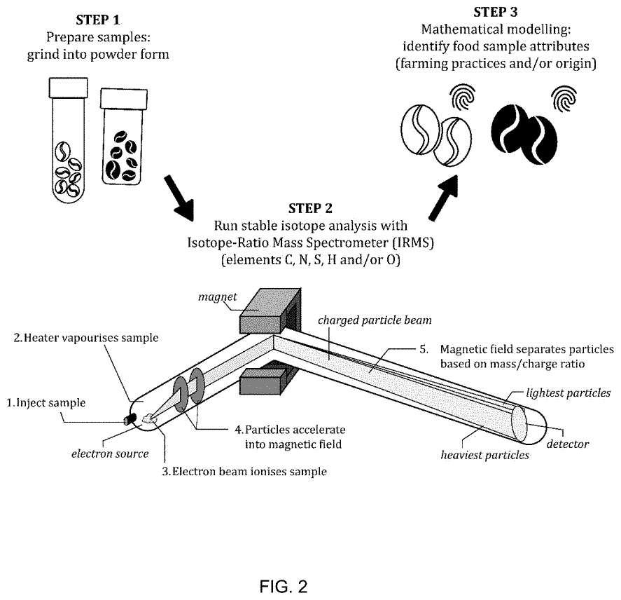

FIG. 3 shows an embodiment of the stable isotopes detectable by the method of FIG. 1 and the corresponding food characteristic deducible from the stable isotopes detected;

FIG. 4 shows the variation in the concentration of stable isotope of carbon (¹³C) in grain-fed cows and grass-fed cows;

FIG. 5 shows the variation in the concentration of stable isotope of nitrogen (¹⁵N) in farmed fish and wild caught fish; and

FIG. 6 is a block diagram showing a system implementing the method of FIG. 1.

FIG. 7 is a graph showing carbon and nitrogen stable isotope values measured in wild caught and farmed salmon samples; and

FIG. 8 is a graph showing δ³⁴S and δ²H values of coffee originating from the countries Columbia and Ethiopia.

### DESCRIPTION OF PREFERRED EMBODIMENTS

The following description is of preferred embodiments by way of example only and without limitation to the combination of features necessary for carrying the invention into effect.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

It should be understood that the elements shown in the figure, may be implemented in various forms of hardware, software or combinations thereof. Preferably, these elements are implemented in a combination of hardware and software on one or more appropriately programmed general-purpose devices, which may include a processor, memory and input/output interfaces.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

Thus, for example, it will be appreciated by those skilled in the art that the diagrams presented herein represent conceptual views of systems embodying the principles of the invention.

The functions of the various elements described or shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (“DSP”) hardware, read-only memory (“ROM”) for storing software, random access memory (“RAM”), and non-volatile storage.

In the claims hereof, any element expressed as any means for performing a specified function is intended to encompass any way of performing that function including, for example, a) a combination of circuit elements that performs that function or b) software in any form, including, therefore, firmware, microcode or the like, combined with appropriate circuitry for executing that software to perform the function. The invention as defined by such claims resides in the fact that the functionalities provided by the various recited means are combined and brought together in the manner which the claims call for. It is thus regarded that any means that can provide those functionalities are equivalent to those shown herein.

The invention generally relates to a method and a system for determining characteristics of a food or a food substance based on stable isotope analysis. Stable isotope analysis measures the natural weight variations of atoms comprising all matters or materials. Such information can be used as fingerprints to identify the respective materials such as food materials. These fingerprints can be determined to identify or to confirm different attributes or characteristics of food, such as but not limited to, farming practices, feed type, and geographical origin, etc. of the biological materials comprising the food. Examples of stable isotopes detectable and could be used to deduce characteristics of food products may include carbon isotopes (¹³C, ¹²C) nitrogen isotopes (¹⁵N, ¹⁴N) oxygen isotopes (¹⁸O, ¹⁶O) hydrogen isotopes (²H, ¹H) and sulphur isotopes (³²S, ³³S, ³⁴S and ³⁶S). A person skill in the art will appreciate that any stable isotopes of other elements forming a food substance in a detectable concentration may also potentially be used to practice the present invention and therefore, the present invention shall not be limited by any specific embodiments herein described or illustrated. Food of plant and/or animal origins are found to reflect attributes or characteristics of their farming environment based on the stable isotope ratio of elements such as ¹³C/¹²C ¹⁵N/¹⁴N, ¹⁸O/¹⁶O and ²H/¹H present in the food substance. For example, the stable isotopes ratio of ¹³C/¹²C are found to vary with geographic location and climate. Plants grown in humid environments take in more carbon dioxide are found to develop a lower ratio of ¹³C/¹²C than plants in drier environments.

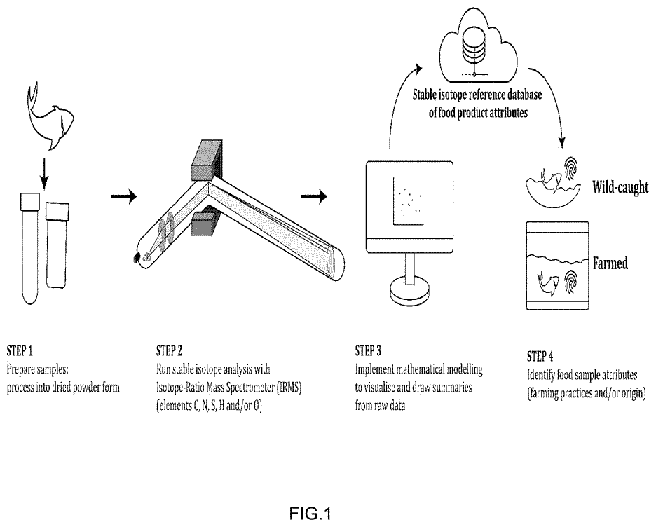

Referring to FIG. 1, shown is a method and a system for determining characteristics of a food substance. In the context of the present invention, the expressions “food”, “food product” and “food substance” are used interchangeably and should be understood in broad term to relate to any food, beverages and/or consumables, either processed or in raw material forms, which may include, or be derived from, any plants, animals, a part thereof and/or a combination thereof. For example, the food substance may include, but is not limited to, wine, honey, olive oil, water, spices, grains, nuts, meat, poultry, fish, vegetable, fruits and tobacco, etc. A person skill in the art will appreciate that any other consumables comprising materials having stable isotopes in a detectable concentration may potentially be tested for one or more attributes or characteristics by the present invention, and therefore, the present invention shall not be limited by any specific food substances described or illustrated.

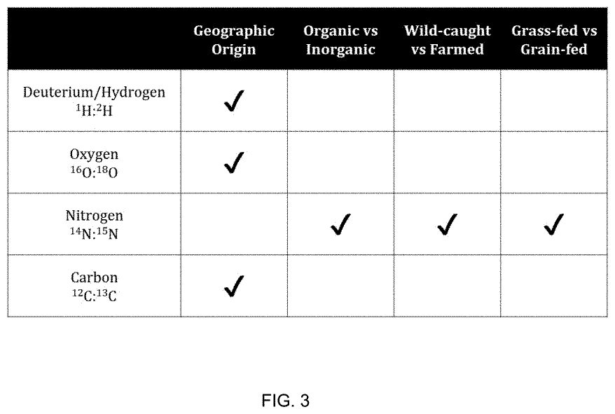

In one embodiment, the method comprises detecting at least one stable isotope of one or more elements of a food substance with unknown characteristic. In the context of the present invention, the term “characteristic” of the food substance should be given a broad meaning which may generally comprises one or more of farming practice, production method, geographic origin, seasonal origin, and environmental condition of the food substance. For example, the characteristic may relate to whether the livestock, e.g. cow, sheep, pig, etc. was grain-fed or grass-fed; whether the poultry e.g. chicken and duck etc. was farmed in cages or free-ranged; whether the seafood is farmed or wild caught in nature; the geographic location where the crops, e.g. grapes for wine, rice, fruit, etc. was grown and the livestock was born, raised and farmed; and whether the food substance is organic or inorganic, etc. An embodied correlation of a mathematical attribute of stable isotopes such as isotope ratios of hydrogen (¹H:²H), oxygen (¹⁶O:¹⁸O), nitrogen (¹³N:¹⁵N) and carbon (¹²C:¹³C) with the corresponding characteristics of foods substance including geographic origin, organic vs inorganic, wild vs farmed, and grain-fed vs grass-fed are shown in FIG. 3. It is found that the light to heavy isotope ratios of these elements vary with geographic and nutritional sources for crops and livestock. For example, organic vegetables are found to have more heavy nitrogen isotope than conventionally farmed vegetables because manure has relatively more heavy nitrogen than synthetic fertilizers. The analysis of light to heavy isotope ratios thus allows characteristics of food such as grass-fed vs. grain-fed beef and wild vs. farmed seafood be distinguishable based on the present invention.

The detection of at least one stable isotope of one or more elements from the food substance can be conducted by an analytic apparatus or instrument such as a mass spectrometer, which measures the relative abundance of isotopes based on mass-to-charge ratio of ions. Preferably, the mass spectrometer is a continuous flow isotope ratio mass spectrometer (IRMS), although the skill person may appreciate that similar instrument or apparatus for the detection and analysis of stable isotopes of elements shall also be applied and encompassed.

Prior to the detection of stable isotopes from the food substance, the food substance should first be prepared or processed in the form of a sample, and more preferably, the sample is provided or prepared in powder form. In one embodiment, the sample can be prepared by one or more steps of drying such as freeze-drying, extracting, cooling and grinding of the food item in ambient condition or under vacuum. For example, a sample may be subjected to low temperature and low pressure to facilitate drying of a wet or liquid sample. Alternatively, a solid sample may be maintained at 60° C. to 70° C. for 24 to 48 hours to fully dry. Following drying, a sample is homogenized using a mortar and pestle or a homogenizer such as a TissueTearor or a beadbeater. Homogenizing a sample through any of these means results in a dry powder, which must be weighed out into small aliquots (1.0 mg to 5.0 mg) in tin capsules, which are then folded to enclose the powder.

Some samples may require lipid extraction. In this case, a frozen or dried sample will be submerged in a corresponding volume of solution, composed of 3 parts hexane to 2 parts isopropanol by volume, or other extraction solvent. Lipids are dissolved into the solvent as the solvent is homogenized with the solution. Centrifugation of the homogenate at 1000 rcf to 1500 rcf for 3 to 5 minutes will separate the extraction solvent containing the dissolved lipids in a distinct liquid layer from the solid layer of the rest of the homogenized tissue. Pouring or pipetting off the liquid fraction and allowing the remaining solvents to evaporate off in a drying oven 60° C. to 70° C. for 24 to 48 hours will result in a lipid-free, dry powder, which may be used in stable isotope analysis.

After the sample is prepared and it will be introduced into the IRMS followed by one or more of atomizing and/or ionizing steps. Preferably, the step of atomizing the sample comprises heating to vaporize the sample into gaseous form, for example, carbon dioxide for food comprising carbon element and nitrogen gas for food comprising nitrogen element. The gases resulting from combustion will be deflected under a magnetic field based on mass. The gas molecules of different weights will be detected with Faraday cup detectors, allowing for the determination of the ratio of stable isotopes present in the vaporized sample.

Signal resulted from the IRMS detection of the stable isotopes will then be processed by a computer device based on mathematical modelling. In one embodiment, the step of processing signal from the detected stable isotopes may comprise mathematical computation based on one or more of the following attributes: ratio of two or more isotopes of the same element such as hydrogen (¹H:²H), oxygen (¹⁶O:¹⁸O) nitrogen (¹³N:¹⁵N), carbon (¹²C:¹³C), or sulfur (³⁴S:³²S), ratio of two or more isotopes of different elements; concentration or mass percentage of at least one stable isotope of one or more elements such as hydrogen, (¹H or ²H), oxygen (¹⁶O or ¹⁸O), nitrogen (¹³N or ¹⁵N) and carbon (¹²C or ¹³C) and/or sulfur (³⁴S:³²S); and relative deviation (δ) of two different isotopes of the same element such as hydrogen (δ²H), oxygen (δ²⁸O), nitrogen (δ¹⁵N), carbon (δ²¹³C) or sulfur (δ³⁴S) relative to a standard. The computation of the relative deviation (δ) may preferably be calculated as:

δX=[(Rₛₐₘₚₗₑ/R_{standard})−1]×10³
where:
X═²H, ²⁸O, ¹³C, ¹⁵N, or ³⁴S
Rₛₐₘₚₗₑ/R_{standard}=ratio of ¹H/²H, ¹⁶O/¹⁸O ¹³C/¹²C, ¹⁵N/¹⁴N, or ³⁴S/³²S in the sample or the standard

In one embodiment, the computation may comprise one or more modellings and/or algorithms.

After the signal from the stable isotopes detected are processed by the computer modelling, the processed signal or data will be analyzed with respect to a standard isotopic profile of the food substance with known characteristic to thereby deduce a characteristic of the food substance being tested. Particularly, the analyzing step comprises comparing one or more of the computed attributes as described above with respect to corresponding attributes of the food substance with a known characteristic profile.

For example, farming practices could have a distinctive stable isotope ratio profile of nitrogen (¹⁴N vs ¹⁵N), carbon (¹²C vs ¹³C) and sulphur (³²S vs ³³S vs ³⁴S vs ³⁶S). The farming practices of the food substance can therefore be identified by comparing against referencing stable isotope profile or portfolio database of said food substance. The isoscape of water, i.e. variations in stable isotopes of hydrogen and oxygen around the world, illustrates the relative deviations of oxygen (δ¹⁸O ‰) and hydrogen (δ²H ‰) with geographic distribution. For example, fruits of different geographic origin, such as Japanese strawberries and Australian avocados, can be determined on the basis that stable isotopes ratio of hydrogen and oxygen in the water molecules in the fruits would be similar to those of water in their respective country of origin according to the geographical isoscape.

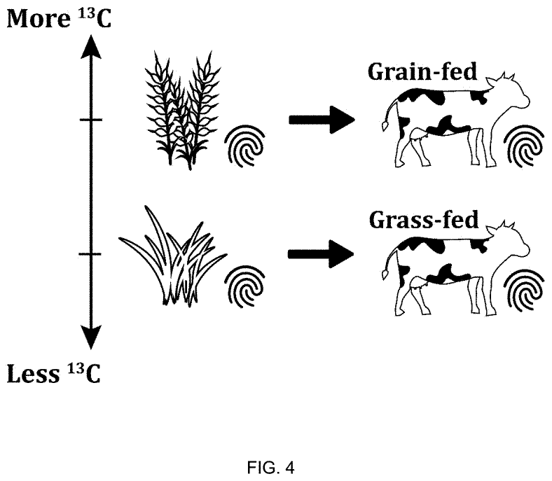

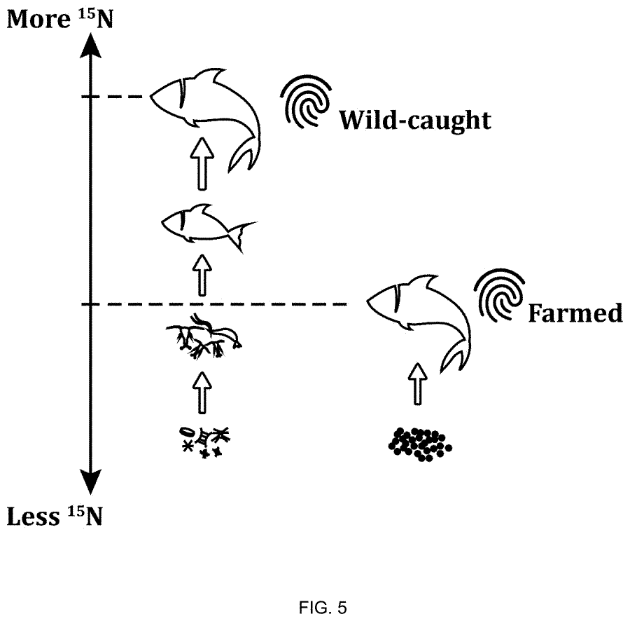

Correlation of concentration of ¹³C stable isotope with respect to the feeding practice, i.e. grass-fed or grain-fed of cows are shown in FIG. 4. Elements in the feed of animals are found to be assimilated into their tissues. By measuring the stable isotope ratios of these elements, the geographical origin and/or attributes of their feed can be deduced to thereby verify production methods of food. For example, a lower ¹³C concentration in the beef may indicate that the cow from which the beef is obtained was grass-fed, whereas a higher ¹³C concentration indicates that the cow was grain-fed. FIG. 5 further illustrates the correlation of concentration of ¹³N stable isotope with respect to the farming practice, i.e. a lower ¹³C concentration indicates that the fish is likely to be a farmed fish whereas a higher ¹³N concentration indicating that the fish is likely to be a wild caught fish.

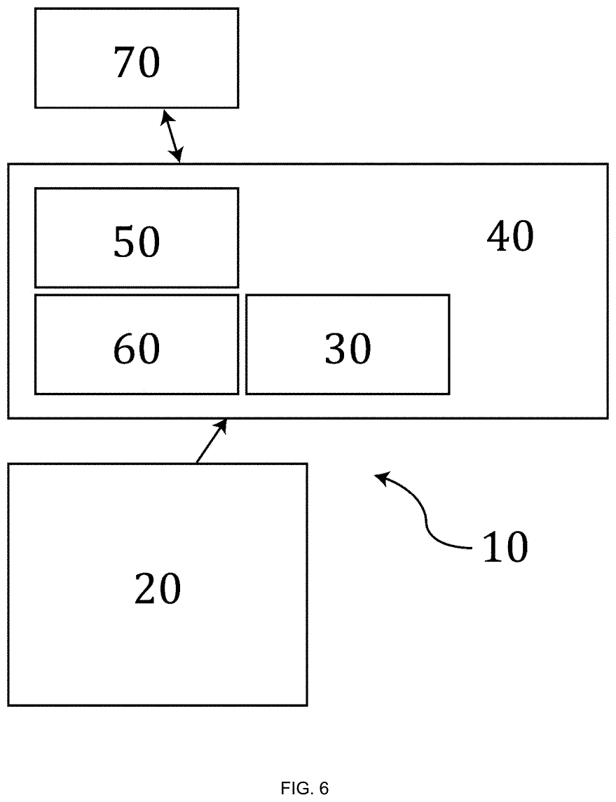

In another aspect the present invention, it relates to a system 10 implementing the steps for determining characteristics of food substance as above-described. As shown in FIG. 6, the system 10 comprises an analytical apparatus 20 such as a mass spectrometer 20 for detecting at least one stable isotope of one or more elements of the food substance. Preferably, the mass spectrometer comprises or being a continuous flow isotope ratio mass spectrometer (IRMS).

The system 10 further comprises a computer device 40 comprising a communication module 30 for receiving detection signal from the mass spectrometer 20. The computer device further comprises a memory 50 for storing data and a processor 60 for executing computer readable instructions. The processor 60 is configured by the computer readable instructions when being executed thereby processes the detected signals received via the communication module 30 from the mass spectrometer 20, and subsequently, analyses the processed signals of the stable isotopes of the elements of the tested food substance with unknown characteristic, with respect to a standard isotopic profile of the food substance with known characteristics thereby deducing a characteristic of the food substance being tested.

In one embodiment, the computer device 40 can be configured as a stand-alone device or a system. Alternatively, the computer device 40 can be provided as a communication equipment, for example, a smart phone, a tablet computer, a desk top computer, a laptop computer, a personal computer (PC), or the like, although any suitable data processing device or system may also be utilized. The computer device 40 may also be provided at, or connected directly or via a network connection to a server 70 and/or a database 70, with the server 70 comprising a local server, a remote server such as a cloud-based server, and/or a distributed network of servers, although this is not essential to the implementation of the invention. The network connection may comprise a wireless communication network such as a wireless cellular network, a wired network, the internet and/or any combination of the foregoing.

In one embodiment, the processor 60 of the computer device 40 is adapted to execute computer readable instruction to thereby implement the method of the present invention. One of more of the processor 60 and the memory 50 and the communication module 30 can be deployed as part of the computer device 40 and there is no limitation to such a deployment configuration according to the concepts of the invention. For example, each of the processor 60, the memory 50 and the communication module 30 may be deployed as respective functional blocks of the computer device that is distinct from, but connected to, the mass spectrometer 20. Each of the memory 50, the processor 60 and the communication module 30 can also be separately implemented using logic circuits and/or executable code/machine readable instructions stored in the memory 50 of the computer device 40 for execution by the processor 60 to thereby perform functions as described herein. For example, the executable code/machine readable instructions may be stored in one or more memories 50, for example, a random access memory (RAM), a read only memory (ROM), a flash memory, a magnetic memory, an optical memory or the like, suitable for storing one or more instruction sets, for example, an application software, a firmware, an operating system, an applets, and/or the like, data such as configuration parameters, operating parameters and/or thresholds, collected data, processed data, and/or the like, etc. The one or more memories 50 may comprise processor-readable memories for use with respect to one or more processors 60 operable to execute code segments of any one or more functional blocks of the computer device 40 and/or to utilize data provided thereby to perform functions of computer device 40, as described herein. Additionally or alternatively, the processor 60 may comprise one or more special purpose processors such as an application specific integrated circuit (ASIC), a field programmable gate array (FPGA), graphics processing unit (GPU), and/or the like configured to perform functions of the computer device 40 as described herein.

### EXAMPLES

The following examples illustrate exemplified processes of the present invention as described above.

### Methodology

Prior to the detection of stable isotopes from food substances, the food substances should undergo preparation and pre-processing in the form of a sample. Such processes are dependent on the nature of the food substances, with the aim of producing a dry, powdered sample.

Microbial activity must first be stopped, to avoid degradation of the sample which alters the ratio of stable isotopes. Unless acquired in such states, the sample must immediately be frozen and/or dried, and maintained constantly at low temperature and/or low humidity except when necessitated by further processing and analyzing. High-water content samples would preferably be subject to low temperature and low pressure for freeze-drying. Meanwhile low-water content samples may be efficiently dried by heating—maintenance at 60° C. to 70° C. for 24 to 48 hours in a drying oven or desiccator is sufficient to remove all moisture. Other methods to extract water may be used, under ambient conditions, low pressure, vacuum, heating, cooling, or other conditions.

As the distribution of stable isotopes may vary across a sample, homogenization is critical for accurate measurements. A sample in its dried solid form may be homogenized by grinding, for example, with a mortar and pestle, manual or electric burr grinder or blade grinder, bead mill homogenizer or other instruments. Otherwise, a sample may be homogenized in its wet form, prior to the aforementioned drying step, for example, by TissueTearor homogenization, bead beating or other methods. These processes finally result in a dry, finely powdered sample.

Other processes may be necessary to maintain consistency between samples. In animal products, fat and muscle tissue in meat have different stable isotope ratios. As the amount of fat varies between samples of meat, the lipid content is extracted to ensure fair comparison between samples. This process is detailed below within the example of salmon meat.

Here we detail the methods for processing two types of food substances as examples for how the general protocol may be modified according to the characteristics and nature of the sample. Firstly, coffee samples are obtained in dry form. Finely ground coffee may be used directly in stable isotope analysis, whereas whole coffee beans are first homogenized by grinding in a manual burr grinder, or other instrument, to obtain a powder. If processed or stored in a humid environment, ground coffee samples may then be dried in a drying oven at 60° C. to 70° C. for 24 to 48 hours. At this point, the samples will be ready for stable isotope analysis.

However, pre-processing samples such as salmon is more involved. Salmon samples are frozen, if not already, cut into small pieces and then homogenized in a small volume of water using a TissueTearor or other instrument. The homogenized samples are promptly frozen, then freeze-dried to obtain a dry powder. Due to variation in fat content between samples, a small mass of freeze-dried sample, e.g. 100 mg, then undergoes lipid extraction with a corresponding volume of solution, e.g. 2.4 mL, composed of 3 parts hexane to 2 parts isopropanol by volume, or other extraction solvent. Lipids are dissolved, for example, into the hexane-isopropanol solution, when the dry sample is homogenized with the solution. Centrifugation of the homogenate at 1000 rcf for 3 minutes will separate the extraction solvent containing the dissolved lipids in a distinct liquid layer from the solid layer of waterless homogenized muscle tissue. The solid layer is then mixed with additional extraction solvent, e.g. 975 uL with 25 uL water added, to remove residual lipids. The mixture undergoes a final centrifugation at 1500 rcf for 5 minutes. Pouring or pipetting off the liquid fraction and allowing the remaining solvents to evaporate off in a drying oven 60-70° C. for 24-48 hours will result in a lipid-free, dry powder. This may then be used in stable isotope analysis.

Subsequent to sample preparation and pre-processing as detailed above, aliquots of dry, powdered sample will be prepared for stable isotope analysis. The aliquot will be 1.0 mg to 5.0 mg in weight, dependent on the abundance of target elements within the sample, to ensure a signal within the isotope ratio mass spectrometer's (IRMS) range of detection. For both coffee and salmon samples, 3 mg to 3.5 mg is used to assess carbon, nitrogen and sulphur isotope ratios. The aliquots will be weighed in tin capsules, which are then folded to enclose the sample. The capsule is then loaded into an Environmental Analyzer coupled to a Stable Isotope Ratio Mass Spectrometer. These instruments combust the samples, converting the sample mass to gaseous form. Any residual moisture and contaminants, are removed, and the purified gasses are drawn through the instrument to a magnetic field, which separates each gas by weight. The gasses that are measured include H₂ for hydrogen, CO₂ for carbon and oxygen, N₂ for nitrogen, and SO₂ for sulfur. The number of molecules with each possible mass, given the different masses of each stable isotope, are counted using Faraday collection cups. The stable isotope ratios of interest for the salmon are carbon and nitrogen, while for the coffee hydrogen, oxygen, and sulfur are of interest.

### Results and Discussion

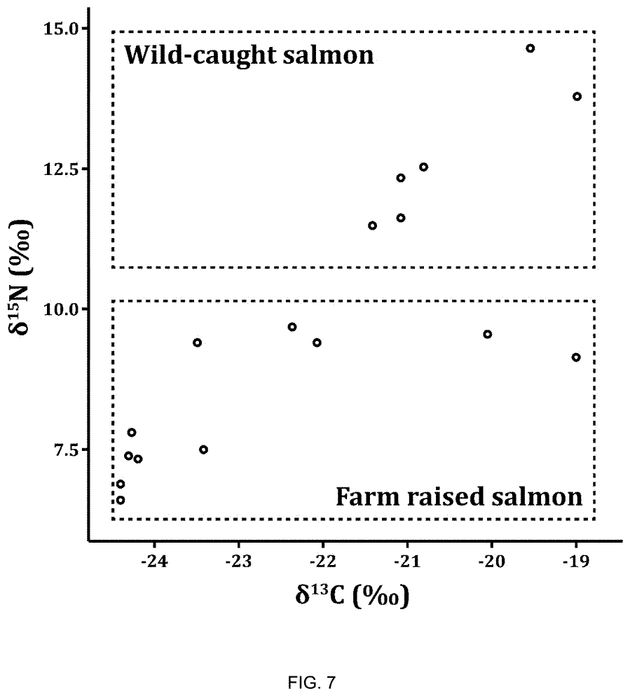

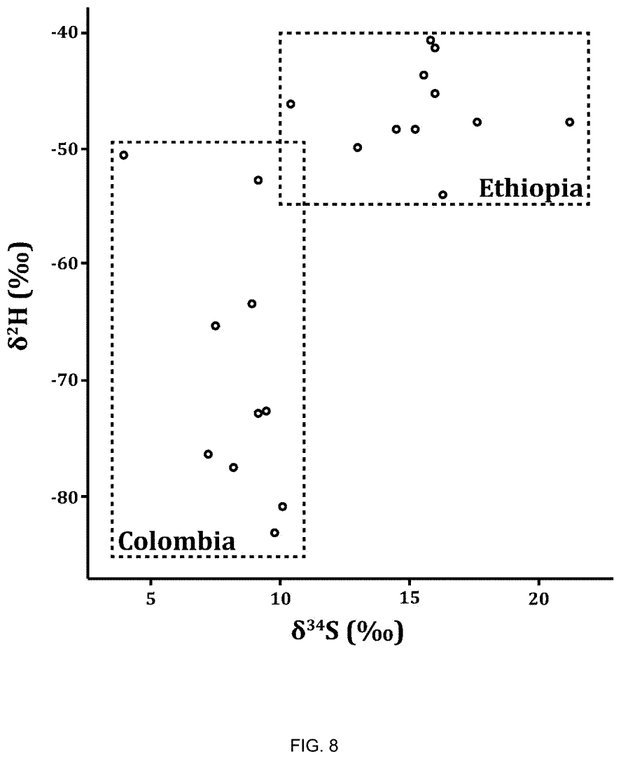

The carbon and nitrogen stable isotope values of salmon samples are indicative of how the salmon was raised and can distinguish between wild caught and farm raised fish. FIG. 7 shows the values of 6 wild-caught salmon and 11 farmed raised salmon. The farm raised salmon have nitrogen isotope values that fall below 10‰ while the wild caught salmon have nitrogen isotope values above 11.5‰. The δ¹⁵N value of salmon tissue can therefore be used to assess whether the fish it came from was wild caught (>11.5‰) or farm raised (<10‰). Sulfur, oxygen, and hydrogen isotope values can be used to determine the geographic origin of food, including coffee. FIG. 8 shows a graph of the δ³⁴S and δ²H values of coffee originating from the countries Columbia and Ethiopia. Coffee from Columbia has lower δ²H values ranging from −83.5 to −52.5‰ whereas Ethiopia has higher values ranging from −40.5 to −53.5‰. While there is some overlap in these ranges, the ratio of sulfur isotopes can be used to further distinguish between the two origins. FIG. 8 shows that coffee from Colombia has lower δ³⁴S ranging from 4.0 to 11.0‰ while coffee from Ethiopia has values ranging from 10.5 to 18.0‰.

The present invention is advantageous in that it provides a system and a method for determining attributes or characteristics of a food or a food substance based on stable isotope analysis. The invention is capable of accurately deducing and thus verifying characteristics of the food being tested such as farming practice, production method, geographic origin, seasonal origin, and environmental condition, etc. For example, whether the livestock was grain-fed or grass-fed, the poultry was kept in cages or free-ranged, the seafood is farmed or wild caught in nature; the region where the crops, e.g. grapes for wine, rice, and fruits etc. were grown and where the livestock, e.g. cow for beef, was born, raised and kept; and whether the food products are organic or inorganic. etc. can be determined by the present invention based on detection and analysis on stable isotopes of elements, such as carbon isotopes ¹³C, ¹²C, nitrogen isotopes ¹⁵N, ¹⁴N, oxygen isotopes ¹⁸O, ¹⁶O hydrogen isotopes ²H, ¹H and sulphur isotopes ³²S, ³³S, ³⁴S and ³⁶S present in the food. The present invention can be widely applicable in almost any food types with no restriction to the geographic origin and/or food processing methods, etc. This is particularly due to the capacity of the present invention in detecting stable isotopes of hydrogen and oxygen in the present invention in addition to other commonly detectable stable isotopes such as carbon and/or nitrogen, as isotope ratios of water vary across the globe according to precipitation patterns and therefore, isoscapes of hydrogen and oxygen can be readily available for comparison. The present invention therefore offers a relatively simple and low cost analytic method and system for food verification and authentical with high accuracy and reproducibility.

The present description illustrates the principles of the present invention. It will thus be appreciated that those skilled in the art will be able to devise various arrangements that, although not explicitly described or shown herein, embody the principles of the invention and are included within its spirit and scope.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

While the invention has been illustrated and described in detail in the drawings and foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method of determining a characteristic of a food substance, comprising steps of:
detecting at least one stable isotope of one or more elements of a food substance with unknown characteristic;
processing signal received from detecting the at least one stable isotope of the one or more elements;
analysing the signal processed with respect to a standard isotopic profile of the food substance with known characteristic thereby deducing a characteristic of the food substance being tested.

2. The method according to claim 1, wherein the at least one stable isotope of the one or more elements is selected from the group consisting of: hydrogen isotopes ¹H and ²H, nitrogen isotopes ¹⁴N and ¹⁵N, carbon isotopes ¹²C and ¹³C, oxygen isotopes ¹⁶O and ¹⁸O, and Sulphur isotopes ³²S, ³³S, ³⁴S and ³⁶S, and a combination thereof.

3. The method according to claim 1, wherein the step of processing signal from the at least one stable isotope of the one or more element comprises computation based on one or more of the following attributes: ratio of two or more isotopes of same element, ratio of two or more isotopes of different elements; concentration or mass percentage of the at least one stable isotope of the one or more elements; and relative deviation (δ) of two different isotopes of same element relative to a standard.

4. The method according to claim 3, wherein the computation comprises one or more modellings and/or algorithms.

5. The method according to claim 1, wherein the step of detecting at least one stable isotope of one or more elements from the food substance is conducted by mass spectrometry.

6. The method according to claim 5, wherein the mass spectrometry comprises continuous flow isotope ratio mass spectrometry (IRMS).

7. The method according to claim 1, wherein the characteristic of the food substance comprises one or more of: farming practice, production method, geographic origin, seasonal origin, and environmental condition.

8. The method according to claim 1 wherein the food substance is of a plant origin, an animal origin, and/or a product derived therefrom.

9. The method according to claim 1, further comprising a step of providing a sample of the food substance prior to the detecting step, wherein the sample is provided in powder form.

10. The method according to claim 9, wherein the step of providing a sample comprising one or more steps of drying, freeze-drying, extracting, cooling and grinding of the food item.

11. The method according to claim 1, wherein the step of detecting at least one stable isotope of one or more elements of the food substance comprises combusting and ionizing the sample.

12. The method according to claim 11, wherein the step of atomising the elements of the sample comprises heating of the sample into gaseous form.

13. The method according to claim 11, wherein the step of ionizing the elements of the sample comprises subjecting the atomized elements under a magnetic field.

14. The method according to claim 9, wherein the step of detecting at least one stable isotope of the one or more elements comprises detecting ionic form of the elements according to mass-to-charge ratio.

15. The method according to claim 3, wherein the step of analysing the processed signal from the at least one stable isotope of the one or more elements with respect to a standard isotopic profile of the food substance with known characteristic comprises comparing one or more of the computed attributes with respect to corresponding attributes of the food substance with a known characteristic profile.

16. A system for determining a characteristic of a food substance, the system implementing the method of claim 1, comprising:
an apparatus for detecting signal of at least one stable isotope of one or more elements of the food substance with unknown characteristic; and
a computer device adapted to receive signal of at least one stable isotope of one or more elements of the food substance detected from the apparatus, the computer device comprising a memory for storing data and a processor for executing computer readable instructions;
wherein the processor is configured by the computer readable instructions when being executed thereby implement steps of processing the signal detected, and analysing the processed signal with respect to a standard isotopic profile of the food substance with known characteristic thereby deducing a characteristic of the food substance being tested.
